(19) United States
(12) Patent Application Publication
(10) Pub. No.: US 2026/0185125 A1
(43) Pub. Date: Jul. 2, 2026

(54) CORN ETHANOL PRODUCTION PROCESS AND METHOD OF PRODUCING CORN ETHANOL

(71) Applicant: Trenton Agri Products LLC, Wichita, KS (US)

(72) Inventor: Joe Shanle, Trenton, NE (US)

(21) Appl. No.: 19/431,511

(22) Filed: Dec. 23, 2025

Related U.S. Application Data
(60) Provisional application No. 63/740,658, filed on Dec. 31, 2024.

(51) Int. Cl. C12P 7/10

(52) U.S. Cl. CPC C12P 7/10

(57) ABSTRACT

In a process for producing corn ethanol, a fermentation ferments corn, a still distills the fermented corn to separate the fermented corn into ethanol and whole stillage, and a dewatering system dewaters the whole stillage. The dewatering system uses a wave separator to separate whole stillage into liquid thin stillage and wet cake. In the wave separator, a driver drives movement of wave generating elements in relation to a filter screen to induce a wavelike motion in the stillage that promotes separating of the liquid thin stillage from the wet cake by breaking surface tension and encouraging the liquid thin stillage to pass through the filter screen.

### CROSS-REFERENCE TO RELATED APPLICATION

This Application claims priority to U.S. Provisional Patent Application Ser. No. 63/740,658, which is hereby incorporated by reference in its entirety.

### FIELD

The present disclosure generally relates to a corn ethanol production process and equipment for dewatering whole stillage.

### BACKGROUND

Referring to FIG. 1, a prior art corn ethanol production process is schematically indicated at reference number 10. The process includes one or more silos 12 that store corn. The silos 12 connect to a mill 14 that mills the corn to produce cornmeal. The cornmeal feeds into a slurry mixer 15, where water recycled from downstream thin stillage is used to wet the cornmeal and form a wetted cornmeal slurry. Enzymes are also mixed into the cornmeal slurry at the mixer 15. The cornmeal slurry is then fed to liquefaction system 16, where the slurry the enzymes are used to convert starches into sugars through enzymatic hydrolysis, thereby producing beer mash. The beer mash is pumped into a beer mash heat exchanger 18, which transfers heat from the hot, liquefied mash to other process streams. After passing through the heat exchanger 18, the mash is cooled in the mash cooler 20 to a temperature suitable for fermentation. The cooled mash is fermented in fermentation vessel(s) 22, and the fermented byproduct (beer) is stored in a beer well 24. The beer well 24 is connected to the beer mash heat exchanger 18 so that the beer absorbs heat from the beer mash before entering the distillation column(s) 26 (broadly, a still). In the illustrated example, the beer mash heat exchanger 18, mash cooler 20, fermentation vessel(s) 22, and beer well 24 cumulatively form a fermentation system for forming beer. In the still 26, the ethanol in the beer is separated from whole stillage. The ethanol from the still 26 is directed into a molecular sieve 28 that dehydrates the ethanol to produce anhydrous ethanol product 30.

The whole stillage from the distillation column(s) 26 is pumped into a whole stillage tank 32 and then to a set of decanter centrifuges 34 that separate the whole stillage into wet cake 36 and thin stillage. The thin stillage is fed into a thin stillage tank 38. A portion of the thin stillage (backset) is recycled back to the slurry mixer 15 to wet the dry cornmeal. The remaining thin stillage is sent to an evaporator 40. The evaporator 40 concentrates the thin stillage by evaporating water to the cook water tank 42. The cook water tank 42 stores water for use in the slurry 15. The remaining liquid in the evaporator forms syrup and is fed into a syrup tank 44. The syrup from the syrup tank is fed into an oil extraction unit 46, which extracts corn oil from the syrup to create corn oil product 48. The material remaining after the oil is extracted and the wet cake 36 produce wet distiller's grains with solubles (WDGS) product 50.

### SUMMARY

In one aspect, a process for producing corn ethanol comprises a fermentation system for fermenting corn. A still is for distilling the fermented corn to separate the fermented corn into ethanol and whole stillage. A dewatering system is for dewatering the whole stillage. The dewatering system comprises at least one wave separator for separating the whole stillage into liquid thin stillage and wet cake. The wave separator comprises a filter screen, wave generating elements, and a driver configured to drive movement of the wave generating elements in relation to the filter screen to induce a wavelike motion in the stillage that promotes separating of the liquid thin stillage from the wet cake by breaking surface tension and encouraging the liquid thin stillage to pass through the filter screen.

In another aspect, a method of making corn ethanol comprises fermenting corn, distilling the fermented corn to separate the fermented corn into ethanol and whole stillage, dewatering the whole stillage in a wave separator to separate thin stillage from wet cake, and extracting corn oil from the thin stillage.

Other aspects and features will be apparent hereinafter.

### BRIEF DESCRIPTION OF THE DRAWINGS

FIG. 1 is a schematic block diagram of a corn ethanol production process of the prior art.

FIG. 2 is a schematic block diagram of an exemplary embodiment of a corn ethanol production process in accordance with the present disclosure;

FIG. 3 is a schematic illustration of a wave separator for dewatering whole stillage in a corn ethanol production process in accordance with the present disclosure;

FIG. 4 is a photograph of an example wave separator with a lid removed to reveal internal components including a whole stillage distributor;

FIG. 5 is a photograph similar to FIG. 4 showing the distributor distributing whole stillage into the wave separator;

FIG. 6 is a schematic illustration showing a shroud connecting the wave separator to a wet cake conveyor;

FIG. 7 is a photograph of the shroud with an access panel removed;

FIG. 8 is another photograph of the shroud with the access panel removed;

FIG. 9 is a photograph of the wave separator supported on a stand in the corn ethanol production process;

FIG. 10 is another photograph of the wave separator and stand;

FIG. 11 is yet another photography of the wave separator and stand;

FIG. 12 is a photograph of the wave separator with the lid installed; and

FIG. 13 is another photograph of the wave separator with the lid installed.

Corresponding reference characters indicate corresponding parts throughout the drawings.

### DETAILED DESCRIPTION

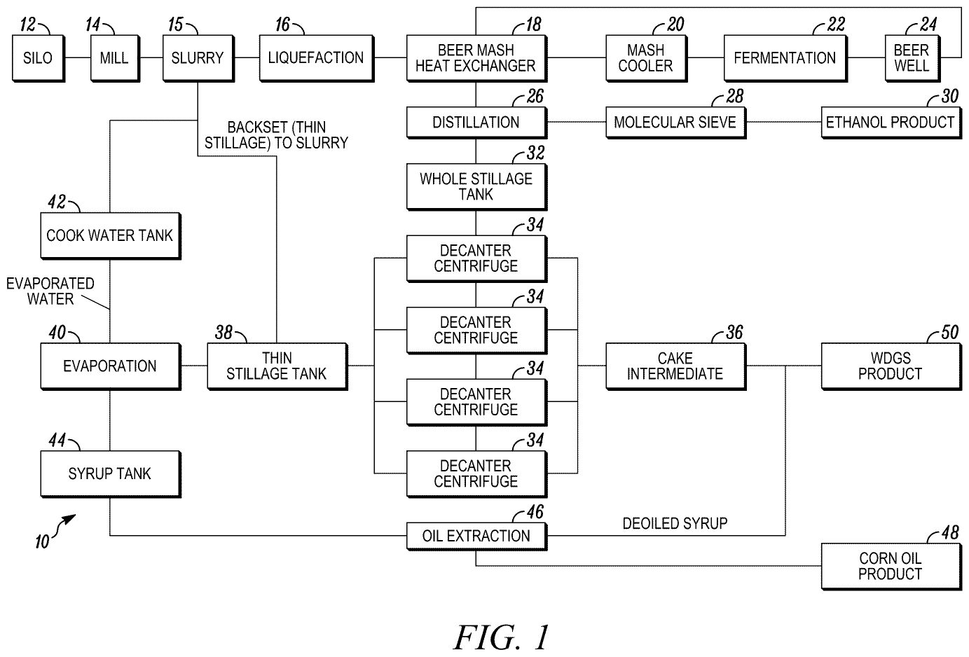

Although the corn ethanol production process 10 depicted in FIG. 1 is very efficient and produces high yields of ethanol product 30, corn oil product 48 and WDGS product 50 from a single integrated process, the inventor has recognized that there are further opportunities to improve the energy efficiency and production output. In particular, the decanter centrifuges 34 that separate the whole stillage into the wet cake 36 and thin stillage are sources of significant energy usage. Conventional decanter centrifuges utilize very powerful (e.g., 200 hp) electric motors and consume about 15% of the electrical energy needed for the entire corn ethanol production process 10. Further, the inventor believes that the decanter centrifuges 34 impart forces on the stillage that can shatter and emulsify the oil droplets. This makes extracting the oil more difficult and reduces the yield of corn oil product 48. The inventor further believes that other downsides to using decanter centrifuges 35 to dewater whole stillage include their substantial maintenance costs and safety concerns. Because decanter centrifuges require very high speed, heavy rotating drums to operate, they require precision maintenance in order to operate safely and reliable. The recurring maintenance adds to the operating costs of the corn ethanol production process 10 and can cause downtime in production.

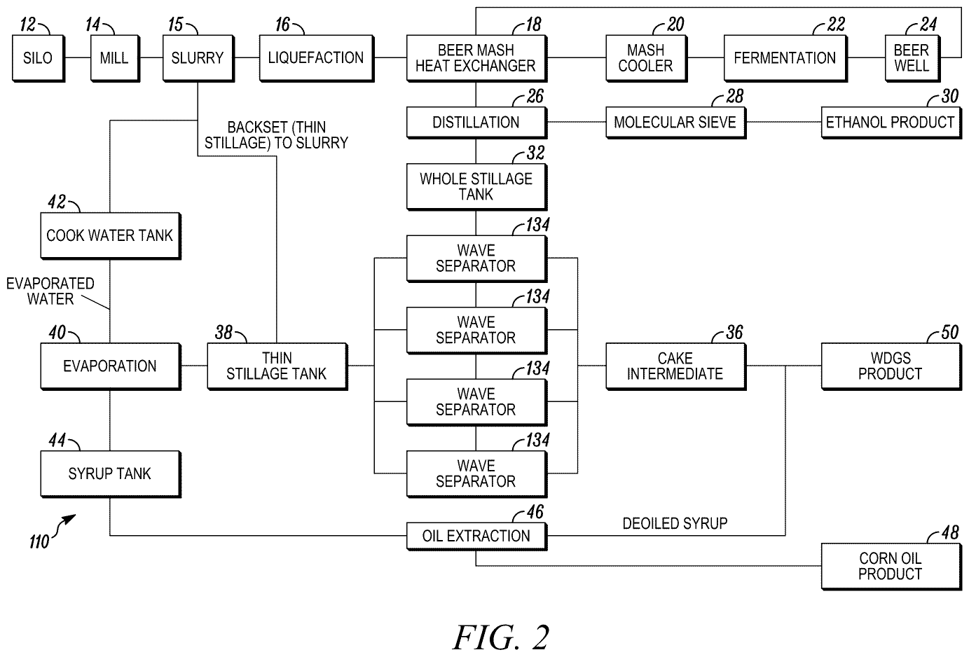

Referring to FIG. 2, to address these problems, the inventor has devised a new corn ethanol production process 110 that replaces some or all of the decanter centrifuges 34 from the conventional corn ethanol production process 10 with wave separators 134. In all other respects, the process 110 is the same as the process 10. In the illustrated process 110, each of the decanter centrifuges 34 has been replaced with a wave separator 134. Hence, the process 110 uses only wave separators 134 to dewater whole stillage. Other corn ethanol production processes 110 in the scope of the present disclosure can employ a combination of wave separators 134 and decanter centrifuges 34 to dewater whole stillage. Further, this disclosure contemplates that wave separators can be used to dewater whole stillage in other types of corn ethanol production processes without departing from the scope of the disclosure.

As will be explained in further detail below, the wave separators 134 use wave generating elements to gently separate liquids from solids and break surface tension, thereby encouraging separation of the liquid thin stillage from the wet cake 36. This gentler process of dewatering the whole stillage does not shatter or emulsify the oil droplets. Moreover, each wave separator 134 requires substantially less power (e.g., less than 10% the horsepower, such as 2 hp instead of 200 hp) to dewater the whole stillage when compared to a conventional decanter centrifuge 34 of comparable throughput. This yields substantial energy savings in the overall corn ethanol production process 110. For example, each wave separator 134 consumes 10%-20% less electricity to dewater the same amount of whole stillage as a comparable decanter centrifuge. Because the dewatering step of any corn ethanol production process is such a substantial consumer of energy in the overall process, these 10-20% savings yield meaningful reductions in global warming potential (GWP) and carbon dioxide equivalents (CO₂e) emissions, resulting in an improved Carbon Intensity (CI) score for the process. The improved energy efficiency also yields improvements in operating margins due to reduced energy costs.

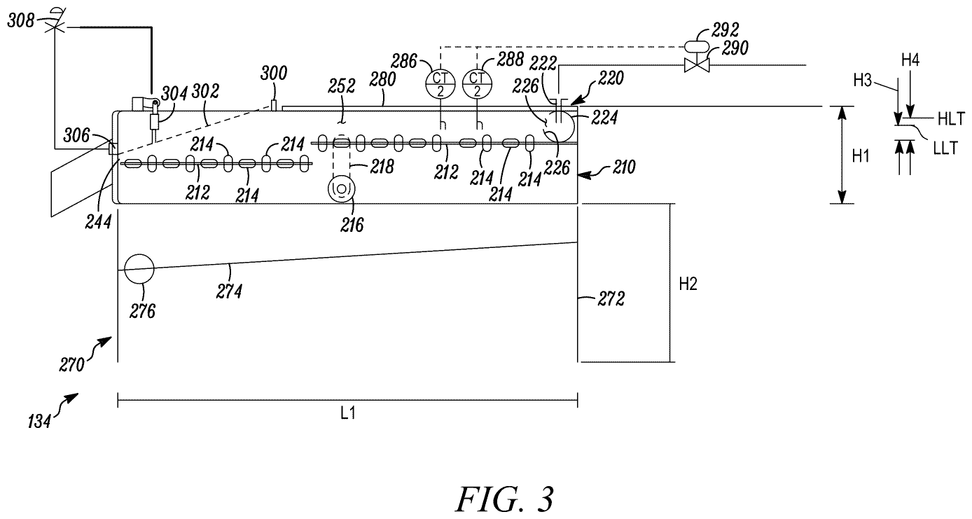

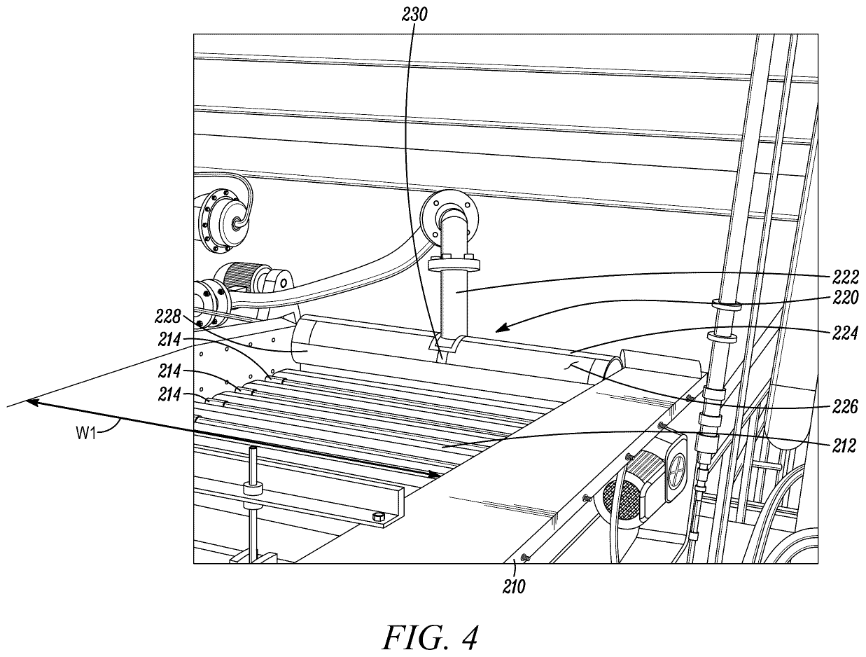

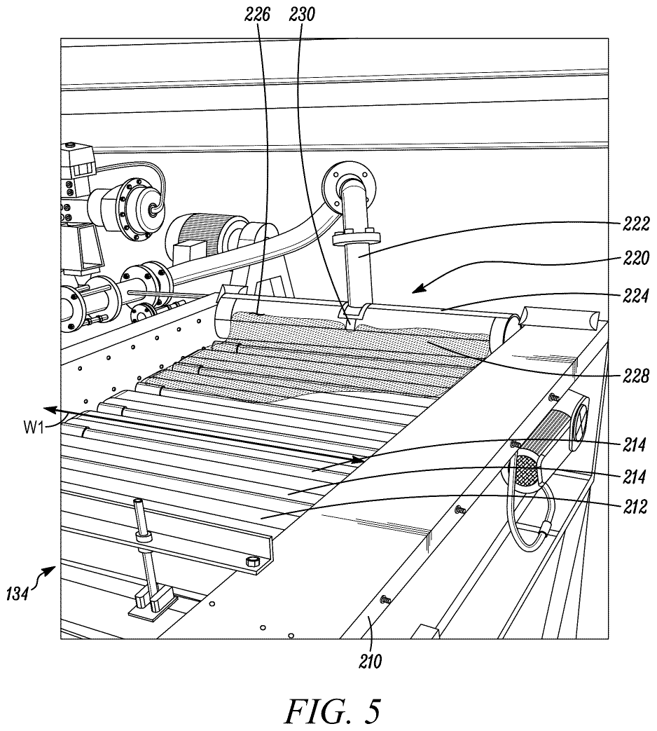

Referring to FIG. 3, an exemplary embodiment of a wave separator 134 in accordance with the present disclosure is shown schematically. The wave separator 134 broadly comprises a housing 210 having an inlet end, an outlet end, a first side wall, a second side wall, a top, a bottom, a length L1 extending from the inlet end to the outlet end, a width W1 (FIGS. 4-5) extending from the first side wall to the second side wall, and a height H1 extending from the bottom to the top. Various types of wave separators may be used to dewater whole stillage without departing from the scope of the present disclosure. In the illustrated embodiment, the wave separator 134 is a Benenv DST-1224, but other wave separators may also be used without departing from the scope of the disclosure.

At least one filter screen 212 is supported on the housing 210. In the illustrated embodiment, there is an upper upstream filter screen 212 and a lower, downstream filter screen 212. Each filter screen 212 extends widthwise between the first and second side walls and lengthwise between the inlet end and the outlet end of the housing 210. Each filter screen 212 is configured to receive whole stillage thereupon (see FIG. 5) such that liquid thin stillage contained in the whole is passable through openings in the filter screen and solid wet cake filters out of the liquid thin stillage to remain on the filter screen.

The wave separator 134 further comprises wave generating elements 214 movable in relation to the filter screen to induce a wavelike motion in the stillage that drives movement of the stillage lengthwise along the filter screen (toward the outlet end). Various types of wave generating elements may be used without departing from the scope of the disclosure, such as oscillating paddles, vibrating plates, or rollers, configured to induce wave-like motion in the mixture. In the illustrated embodiment, the wave generating elements 214 comprise rows of oval discs, with each set of discs in a given row being mounted on a common drive shaft.

The wave separator 134 further comprises a driver 216 configured to drive movement of the wave generating elements 214 to induce the wavelike motion in the stillage. In one or more embodiments, the driver 216 is an electric motor. In certain exemplary embodiments, the driver 216 is a variable frequency drive motor that is able to selectively adjust the speed at which the wave generating elements 214 rotate. In suitable embodiments, the driver 216 is rated at 10 hp or less, 8 hp or less, 5 hp or less, 3 hp or less, or about 2 hp. Accordingly, the wave separator 134 requires substantially less power to dewater whole stillage than an industrial decanter centrifuge.

In the illustrated embodiment, the driver 216 is operably connected to the wave generating elements 214 by a linkage 218 (e.g., one or more chains and sprockets or one or more belts and pulleys). When the driver 216 is activated, it drives movement of the linkage 218, which in turn causes each drive shaft to rotate about a respective axis of rotation. As shown in FIG. 3, the oval discs 214 in successive rows have different angular orientations. The driver 216 and linkage 218 rotate the oval discs 214 in synchronized fashion such that the successive rows of oval discuss induce a gentle, undulating wave motion in the stillage that urges the stillage toward the outlet end of the housing 212 (e.g., in the right-to-left direction in FIG. 3). The motion encourages the liquid thin stillage to fall through the screens 212, while solid wet cake is retained on the surface of the screens until it is discharged through the outlet end of the housing 212.

Referring to FIGS. 4 and 5, to connect each wave separator 134 to the still 26 (e.g., via the whole stillage tank 32) such that whole stillage from the corn ethanol production process 110 can be imparted into the wave separator 134, the illustrated wave separator 134 is fitted with a high-throughput whole stillage distributor 220. The distributor 220 comprises an inlet tube 22 extending vertically and an outlet tube 224 extending widthwise between the first side wall and the second side wall of the wave separator housing 210. The outlet tube 224 defines an outlet opening 226 and comprises a weir 228 below the outlet opening. The outlet opening 226 and the weir 228 extend widthwise. The distributor 220 is configured to be fluidly connected to the still 26 for receiving whole stillage into the inlet tube 222. The distributor 220 is broadly configured so that whole stillage flows downward through the inlet tube 222 and forcefully impacts the outlet tube 224 to create turbulent flow, after which time, the whole stillage backfills behind the weir 228 until it overflows through the outlet opening 226 onto the filter screen 212 at a substantially even flow rate widthwise along the filter screen.

In certain exemplary embodiments, the distributor 220 is configured to accept whole stillage imparted into the inlet tube 222 at a flow rate in an inclusive range of from 25 gal/min to 150 gal/min. The outlet tube 224 can suitably comprise an inner diameter in an inclusive range of from 70 mm to 130 mm. At any flow rate in this range, the distributor 220 disclosed herein can effectively distribute all of the incoming whole stillage to the wave separator 134. Effective distribution requires good mixing of solids and liquid. This is achieved by the turbulence created when the whole stillage impacts the outlet tube 224 after it flows into the outlet tube from the inlet tube 222 in a direction transverse to the span of the outlet tube. Effective distribution also requires a relatively even discharge of the (well-mixed) whole stillage across the filter screen. This is achieved by creating backflow behind the weir 228 before discharging the whole stillage through the outlet opening 226.

In the illustrated embodiment, the filter screen 212 has width corresponding to the inside width W1 of the housing 210. Suitably, the outlet opening 226 has a total width extending from a first end to a second end thereof that is at least 85% of the screen width. This enables the distributor 220 to distribute the whole stillage across essentially the full width of the filter screen 212 at a substantially evenly distributed flow rate.

Referring again to FIG. 3, the outlet tube 224 is cylindrical and, in the orientation shown in the schematic illustration, has a circumference including a six o'clock position at a bottom of the outlet tube, a twelve o'clock position at a top of the outlet tube (the inlet tube 222 extends upward at the twelve o'clock position), a nine o'clock position at a point nearest the outlet end along the length L1 of the housing 210, and a three o'clock position at a point nearest the inlet end along the length of the housing. In the illustrated embodiment, the weir 228 is sized and arranged so that the weir has a top edge that is circumferentially located between a seven o'clock position and the nine o'clock position. The outlet opening 226 extends circumferentially from the top edge of the weir to an opposite edge circumferentially located between a ten o'clock position and a one o'clock position.

Referring again to FIGS. 4 and 5, in one or more embodiments, the distributor 220 further comprises a splitter 230 in a path of the whole stillage flowing downward between the inlet tube 222 and the outlet tube 224. The splitter 230 has a first (e.g., left) side and a second (e.g., right) side and is configured to split the whole stillage from the inlet tube 222 into a first flow stream on the first side of the splitter and a second flow stream on the second side of the splitter. This aids in distributing the whole stillage along the full width of the filter screen 212.

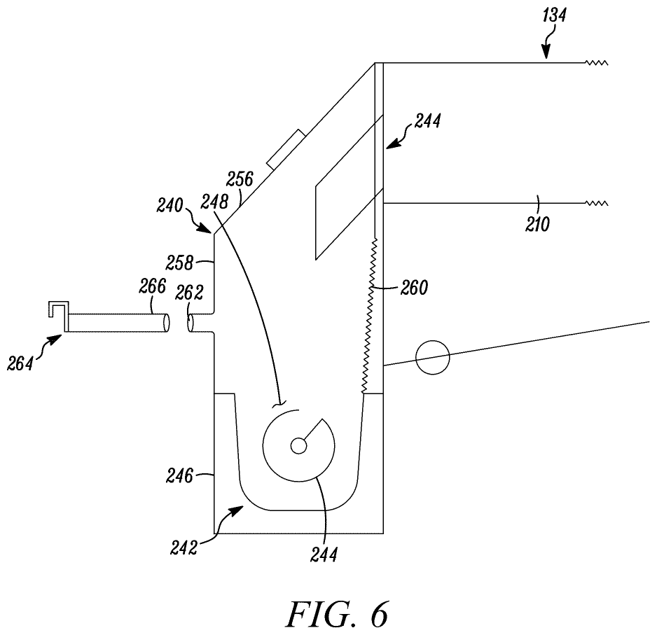

Referring to FIGS. 6-8, the outlet end of the wave separator 134 is suitably fitted with a shroud 240 configured to connect the outlet end to an enclosed wet cake conveyor 242 of the corn ethanol production process 110. The shroud 240 is broadly configured to direct wet cake discharged through an outlet opening 244 in the outlet end of the housing 210 into the wet cake conveyor 242 and isolate the wet cake from ambient environment external to the wave separator housing, the shroud, and the enclosed wet cake conveyor.

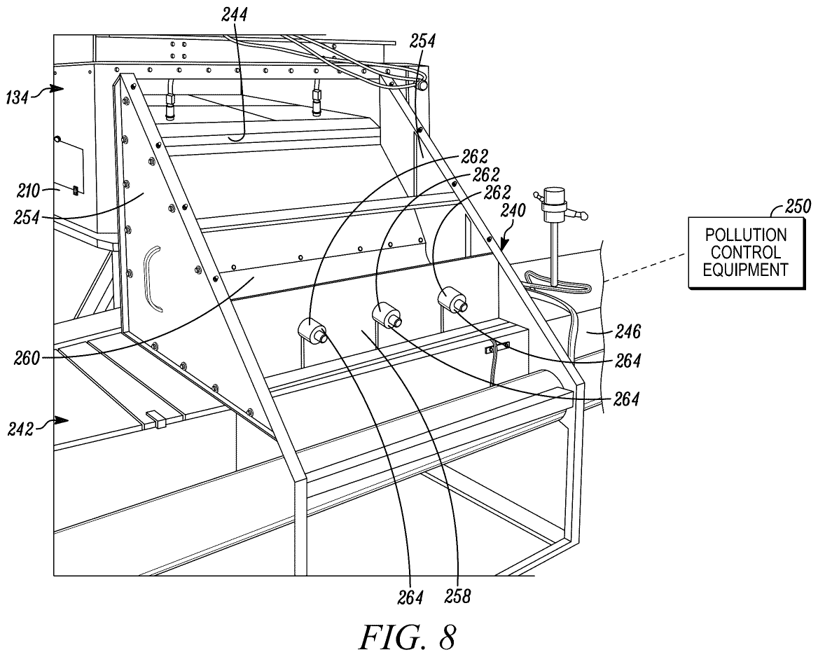

In the illustrated embodiment, the wet cake conveyor 242 comprises a screw conveyor 244 contained in a duct 246. An opening 248 is formed in the top of the duct adjacent the outlet end of the wave separator housing 210. The opening 248 is shaped and arranged so that wet cake discharged through the outlet opening 244 of the wave separator 134 falls into the wet cake conveyor 242 through the opening, and the screw conveyor 244 subsequently conveys the wet cake material to a downstream processing station. Referring to FIG. 8, in the illustrated embodiment, the wet cake conveyor 242 is in fluid communication with pollution control equipment 250. The pollution control equipment 250 imparts a vacuum in the duct 246 that draws out gasses that may contain volatile organic compounds or other pollutants. Notably, the shroud 240 is configured to fluidly connect an interior space 252 (FIG. 3) inside the wave separator to the wet cake conveyor 242 such that the negative pressure inside the duct 246 draws out gasses from the interior space 252 into the pollution control equipment 250.

Referring again to FIGS. 6-8, the shroud 240 comprises first and second side walls 254 spaced apart along the width W1 of the separator. Each of the first side wall 254 and the second side wall 254 comprises a rear edge margin joined to the outlet end of the wave separator housing 210 and a bottom edge margin joined to the top of the duct 246 of the enclosed wet cake conveyor 242. Each side wall 254 also includes an upper edge margin sloping downward and away from the outlet end of the wave separator housing 210.

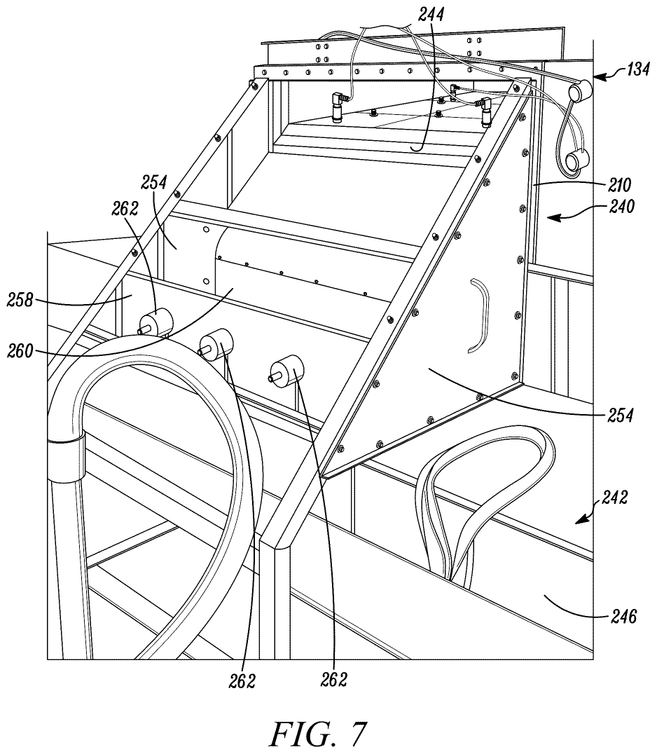

As shown in FIG. 6, the shroud 240 comprises an access panel 256 extending from the upper edge margin of the first side wall 254 to the upper edge margin of the second side wall 254. The access panel 256 is selectively removable or openable to provide access to the interior of the shroud. FIGS. 7 and 8 show the shroud 240 with the access panel 256 removed.

The shroud 240 further comprises a sampling panel 258 extending widthwise between the first side wall 254 and the second side wall 254 and height wise from a lower edge margin joined to the enclosed wet cake conveyor 242 to an upper end margin configured to meet the access panel 256. As will be explained in further detail below, the sampling panel 258 is configured to facilitate in-process sampling of the wet cake being discharged from the wave separator 134.

The shroud 240 further comprises a back panel 260 extending widthwise between the first side wall 254 and the second side wall 254 and height wise from an upper edge margin joined to the outlet end of the wave separator housing 210 below the outlet opening 244 to a lower edge margin joined to the wet cake conveyor 242. In the illustrated embodiment, the back panel 260 is formed of flexible material, more particularly, a rubber material.

The shroud 240 suitably comprises one or more sample ports 262 in the sampling panel 258 at spaced apart locations along a width W1 of the wave separator 134. In the illustrated embodiment, there are three sample ports 262 at spaced apart locations along the width W1 of the wave separator 134. But it will be understood that other embodiments can use other numbers and arrangements of sample ports without departing from the scope of the disclosure. The shroud 240 further comprises a plurality of removable sampling tubes 264. Each sample port 262 has one of the sampling tubes 264 removably received therein. As shown in FIG. 6, each sampling tube 264 has a proximal end portion a distal end portion and a sampling inlet 266 adjacent the distal end portion. When each sampling tube 264 is received in the respective sample port 262, the sampling tube is configured to close the respective sample port and to receive a sampling of the wet cake discharged from the outlet opening into the sampling tube through the sampling port 266. During use, each sampling tube 264 is configured to be removed from the sampling port 262 whereby a sample of the wet cake discharged from the outlet opening 244 at the widthwise location of the sampling port can be removed from the shroud in-process. This allows the operator to inspect the condition of the wet cake in-process, so that any necessary process adjustments relating to the quality of the wet cake can be made in real time.

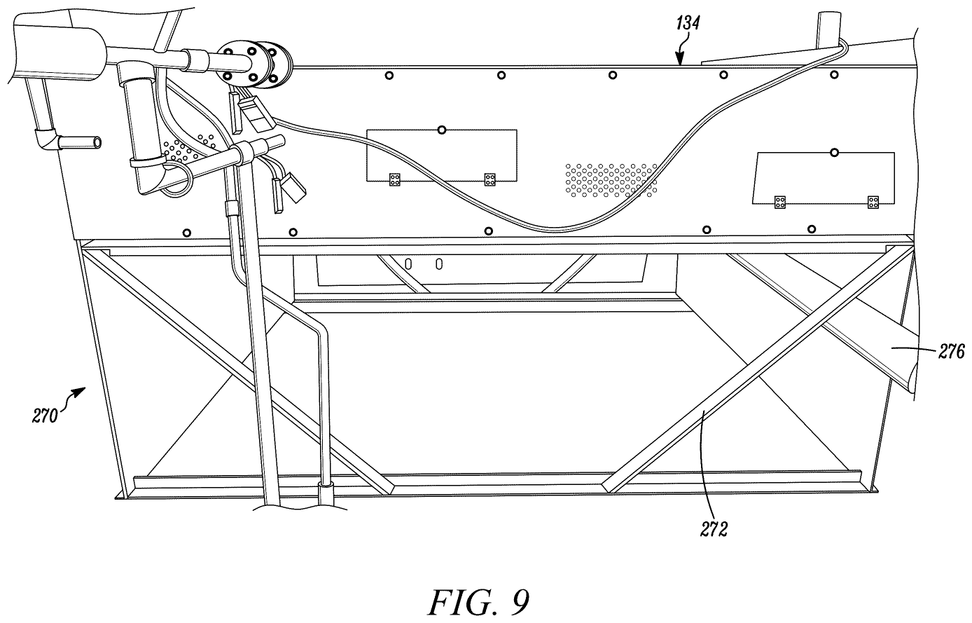

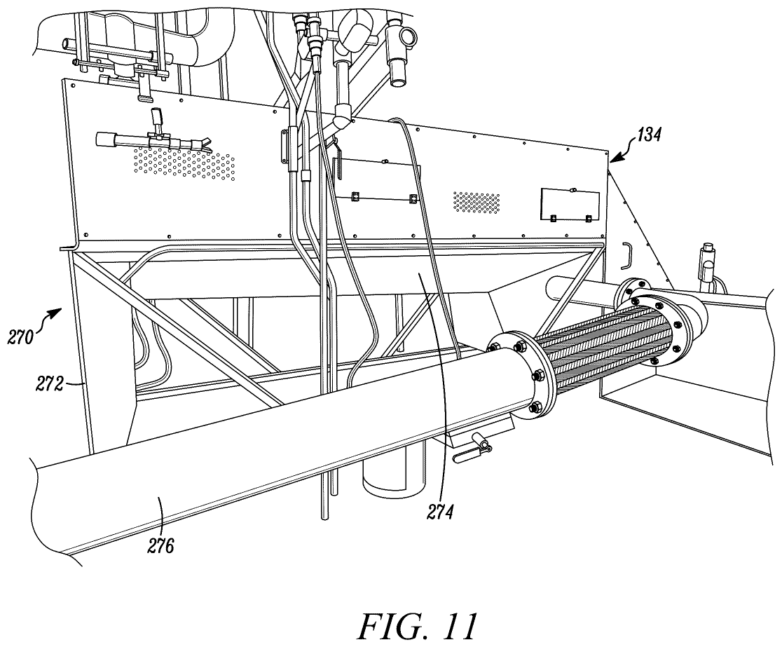

Referring to FIGS. 9-11, in the illustrated corn ethanol production process 110, the wave separator 134 is supported at an elevated position above an underlying support surface (e.g., a floor) on a stand 270. The stand has a height H2, and in one or more embodiments, the height H2 of the stand is of at least 800 mm. The stand 270, thus, elevates the wave separator 134 so that the outlet opening 244 is above the wet cake conveyor 242, which allows the wet cake material discharged through the outlet opening 244 to fall into the wet cake conveyor by force of gravity. The stand 270 comprises a framework 272 that supports the wave separator 134 and a gutter 274 supported on the framework below the filter screen 212. As explained in further detail below, the gutter 274 is broadly configured to catch the liquid thin stillage that passes through the filter screen 212 during dewatering and direct the thin stillage into a thin stillage line 276 of the corn ethanol production process 110 that connects to the thin stillage tank 38 and/or other downstream thin stillage processing stations.

The gutter 274 has an upstream end portion below the inlet end portion of the wave separator 134 and a downstream end portion below the outlet end portion of the wave separator. The upstream end portion of the gutter 274 is elevated above the downstream end portion, as shown in FIG. 3. In one or more embodiments, the upstream end portion of the gutter is at least 100 mm above the downstream end portion. The gutter 274 slopes downward lengthwise from the upstream end portion to the downstream end portion such that the gutter is configured to receive thin stillage passing through the filter screen and gravity feed the thin stillage lengthwise to the downstream end portion. In certain embodiments, the gutter 274 has an average incline along its entire length in an inclusive range of from 3% to 12%.

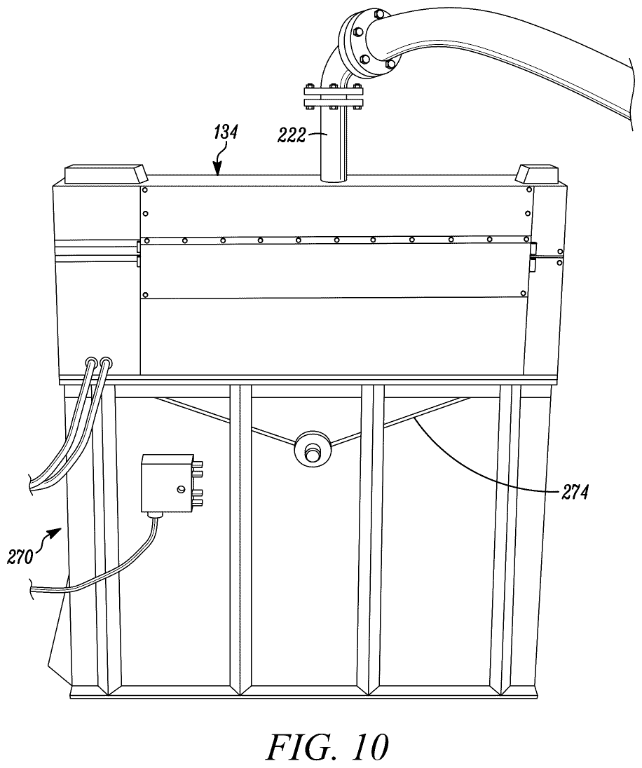

In the illustrated embodiment, the gutter 274 has a V-shaped cross-sectional shape (see FIG. 10). Thus, the gutter 274 is configured to gather the thin stillage in the trough of the V-shaped gutter. The trough of the V-shaped gutter 274 opens into the thin stillage line 276 at the downstream end portion of the gutter. Hence, the gutter 274 is fluidly connected to the thin stillage 276 line at the downstream end portion of the gutter such that the thin stillage gravity fed to the downstream end portion of the gutter is imparted into the thin stillage line. The thin stillage line, in turn, carries the thin stillage to downstream stages of the corn ethanol production process 110 as described above.

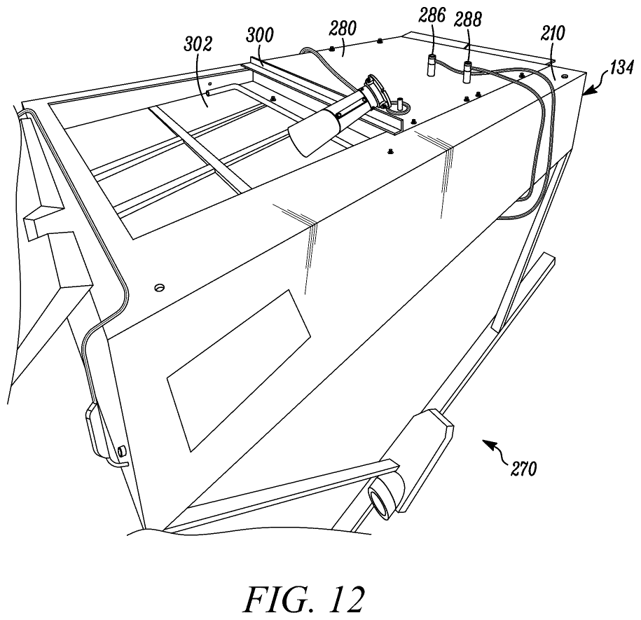

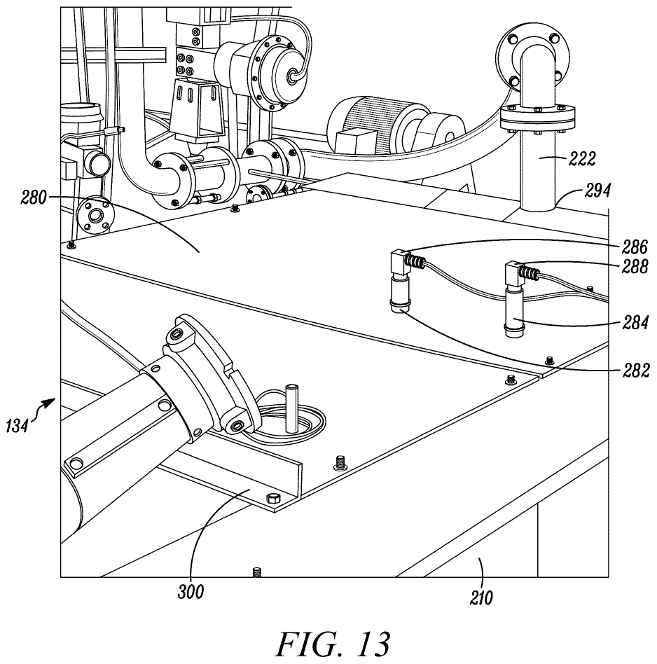

Referring to FIGS. 12-13, the illustrated wave separator 134 further comprises a lid 280 on the top of the housing 210 and enclosing an upper end of a space 252 (FIG. 3) inside the housing such that gasses inside the space are prevented from escaping the space through the top of the housing. As explained above, the outlet end of the wave separator 134 is fluidly connected to pollution control equipment 250 of the corn ethanol production process 210 (via the shroud 240 and wet cake conveyor 242). The negative pressure at the outlet end of the wave separator 134 is communicated through the outlet opening 244 to the space 252 such that gasses inside the space are carried to the pollution control equipment 250 for mitigation.

In the illustrated embodiment, the lid 280 comprises a first sensor fitting 282 and a second sensor fitting 284. The wave separator comprises a low level contact sensor 286 installed in the first sensor fitting 282 and a high level contact sensor 288 installed in the second sensor fitting 284. Each contact sensor 286, 288 can suitably comprise a conductivity fork sensor. The contact sensors 286, 288 together form a level sensing system that is configured to detect a level of stillage in the wave separator 134. Other embodiments can use other level sensor configurations to provide a level sensing system for detecting the stillage level without departing from the scope of the disclosure.

As shown in FIG. 3, the low level contact sensor 286 is configured to detect when stillage reaches a low level threshold LLT and the high level contact sensor is configured to detect when stillage reaches a high level threshold HLT. In an exemplary embodiment, the low level threshold LLT is spaced apart above the nearest filter screen 212 by a first height H3 in an inclusive range of from 75 mm to 100 mm and wherein the high level threshold HLT is spaced apart above the nearest filter screen by a second height H4 in an inclusive range of from 125 mm to 150 mm.

Referring still to FIG. 3, in the illustrated embodiment, the corn ethanol production process 110 comprises a flow control valve 290 between the still 26 (in this case, the whole stillage tank 32) and the wave separator 134 (specifically, the distributor 220 of the wave separator). The dewatering system of the corn ethanol production process 110 further comprises a flow controller 292 configured to control the flow control valve 290 based on the level of whole stillage in the wave separator detected by the contact sensors 286, 288 (broadly, based on the level of whole stillage detected by any suitable level sensing system). When the high level contact sensor 288 detects stillage at the high level threshold, the flow controller 292 automatically closes the flow control valve 290 in order to prevent the wave separator 134 from overflowing. When the outputs from the contact sensors 286, 288 detect stillage in the wave separator 134 at the low level threshold LLT and not the high level threshold HLT (that is, when the low level contact sensor outputs a signal but not the high level contact sensor), the flow controller 292 partially opens the flow control valve 290 (e.g., opens the flow control valve 50%). When there is no stillage detected at even the low level threshold LLT, the flow controller 292 fully opens the flow control valve 290.

Referring again to FIGS. 3 and 12-13, the wave separator 134 further comprises a bracket 300 mounted on the top of the housing 210. The bracket 300 extends widthwise from the first side wall to the second side wall of the housing and is located lengthwise at a location spaced apart from the outlet end toward the inlet end. The lid 290 extends from the inlet end of the wave separator housing 210 to the bracket and covers the top of the wave separator along the entire lengthwise segment extending from the inlet end to the bracket. As shown in FIG. 13, the lid 290 includes a portion covering the distributor 220 such that the inlet tube 222 of the distributor protrudes vertically through the lid. The portion of the lid 290 that covers the distributor 220 includes a central opening 294. The inlet tube 222 extends through the central opening 294, and sealant is applied in the central opening to provide a fluid seal between the inlet tube and the lid 290.

The illustrated wave separator 134 comprises a press plate 302 hingedly connected to the bracket 300 and extending from the bracket to the outlet end of the housing 210. The press plate slopes downward from the bracket 300 to the outlet end. The gap between the lower/downstream end of the press plate 302 and the filter screen 212 forms the outlet opening 244 of the wave separator 134. An actuator 304 (e.g., a pneumatic cylinder) is connected between the housing 210 and the press plate 302 for pressing the press plate toward the filter screen 212. In this way, the press plate 302 is configured to compress the wet cake and wring out some of the remaining moisture immediately before the wet cake is discharged from the outlet opening 244 into the shroud 240.

Referring to FIG. 3, the illustrated wave separator 134 further comprises a moisture sensor 306 (e.g., an infrared moisture sensor) configured to detect an amount of moisture in the wet cake discharged through the outlet opening 244 of the wave separator. The wave separator further comprises a pressure regulator 308 configured to adjust the actuator 304 to control the pressure that the press plate 302 imparts on the wet cake based on the amount of moisture detected by the moisture sensor. For example, if the moisture sensor 306 detects a moisture level that is greater than desired, the pressure regulator 308 adjusts the actuator 304 to impart greater pressure to wring out more liquid from the wet cake prior to discharge through the outlet opening 244.

An exemplary method of corn ethanol production in accordance with the present disclosure will now be described. During use of the corn ethanol production process 110, the fermentation system 18, 20, 22, 24 ferments corn and the still 26 distills the fermented corn to separate the fermented corn into ethanol and whole stillage. The whole stillage is fed (via whole stillage tank 32) to one or more wave separators 134 for dewatering.

The whole stillage flows from a pipe or hose into the inlet tube 222 of the distributor 220. The distributor 220 causes the whole stillage to forcefully impact the outlet tube 224 and divides the flow of whole stillage between two flow streams on opposite sides of the splitter 230. This induces turbulence in the whole stillage to mix liquids and solids and causes the mixed stillage to backfill behind the weir 228 until it overflows through the outlet opening 226 onto the filter screen 212 at an evenly distributed flow rate across the width of the filter screen.

Simultaneously, the driver 216 drives the wave generating discs 214 to rotate in relation to the filter screens 212 to induce a wavelike motion in the stillage on the filter screens. The wavelike motion breaks surface tension and encourages liquid thin stillage to pass through the filter screen onto the gutter 274. The rotating discs 214 also progressively advance the stillage along the length of the wave separator 134 toward the outlet opening 244. Most of the liquid thin stillage that is collected from the whole stillage passes through the filter screens 212 along the upstream portion of the filter screen. Less and less liquid passes out of the stillage the further the stillage progresses along the length of the filter screens 212 toward the outlet opening 244.

While the wave separator 134 is dewatering the stillage, the level sensing system, 286, 288 monitors the level of stillage in the device. When the level of stillage rises above the low level threshold LLT, the low level sensor 286 outputs a signal to the flow controller 292, which adjusts the flow control valve 290 to a partially open state. When the level of stillage further rises further to the high level threshold HLT, the high level sensor 288 outputs a signal to the flow controller 292, which closes the flow control valve. When the level of stillage subsequently falls below the high level threshold HLT, the high level sensor 288 ceases outputting the signal to the flow controller 292, which responds by partially opening the flow control valve 290. When the level of stillage subsequently falls below the low level threshold LLT, the low level sensor 286 ceases outputting the signal to the flow controller 292, which responds by fully opening the flow control valve.

Immediately before the solid wet cake is discharged through the outlet opening 244, the actuator 304 and press plate 302 press the wet cake against the filter screen 212 to wring out some of the remaining liquid. In exemplary methods in accordance with the present disclosure, the moisture content of the wet cake that is discharged from the outlet opening 244 is monitored, either using the moisture sensor 306 or by using the sampling tubes 264 to periodically sample the wet cake as it is discharged. When an integrated moisture sensor 306 is used, the moisture sensor 306 sends information about the measured moisture to the pressure regulator 308, which adjusts the pressure imparted by the actuator 304 to achieve the desired flow rate. When the cake is sampled by hand using the sampling tubes 264, the pressure adjustments to achieve the desired moisture content may be controlled by the operator. In one or more embodiments, the process is controlled to achieve a wet cake moisture content in an inclusive range of from 28% dry matter to 38% dry matter.

The shroud 240 directs wet cake discharged from the outlet opening 244 into the wet cake conveyor 242, which in turn carries the wet cake downstream for use as WDGS product. Meanwhile the pollution control equipment 250 maintains a negative pressure in the wet cake conveyor 242 so that gasses inside the wave separator 134 and shroud 240 are drawn into the pollution control equipment for scrubbing. Throughout the use of the corn ethanol production process 110, the shroud 240 and the lid 280 isolate gasses emitted during the dewatering process from the ambient environment.

Thin stillage that passes through the filter screens 212 is caught by the gutter 274 that is incorporated into the stand 270. The gutter directs the thin stillage into the thin stillage line, which then carries the thin stillage downstream for dehydration through the evaporators to make syrup (and subsequent corn oil extraction) and use in wetting dry cornmeal to create a slurry.

As can now be seen this disclosure, provides a novel corn ethanol production process that incorporates one or more wave separators to dewater whole stillage. The wave separators replace decanter centrifuges in the corn ethanol production process and thereby substantially reduce the energy consumption of the process. Further, the wave separators gently dewater the stillage, protecting the valuable corn oil droplets contained therein for subsequent harvesting. Additionally, whereas conventional corn ethanol production processes including decanter centrifuges yield thin stillage that primarily contains floatable solids, the wave separators disclosed herein yield thin stillage that primarily contain settleable solids. Further, the gentle mechanism of action in the wave separators carries a lower maintenance burden than decanter centrifuges of comparable throughput. The gentle wave action driven by a relatively low-powered driver is also believed to be safer to operate than an industrial decanter centrifuge.

As explained above, a wave separator in accordance with the present disclosure can be equipped with a distributor for imparting well-mixed, well-distributed whole stillage into the device at high flow rates; a level sensing system and flow controls that enable substantially continuous automated operation of the wave separator; a stand that supports the wave separator above a wet cake conveyor and includes integrated guttering for channeling liquid thin stillage downstream in the process; a shroud that encloses the outlet end of the wave separator and guides wet cake discharge into the wet cake conveyor; and/or a lid that mounts the level sensing system and, together with the shroud, contains gasses emitted by the stillage inside the equipment so that they are routed to pollution control equipment for scrubbing rather than being emitted to ambient atmosphere.

Having described the invention in detail, it will be apparent that modifications and variations are possible without departing from the scope of the invention defined in the appended claims.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above products without departing from the scope of the invention, it is intended that all matter contained in the above description and shown in the accompanying drawings shall be interpreted as illustrative and not in a limiting sense.

1. A process for producing corn ethanol, the process comprising:
a fermentation system for fermenting corn;
a still for distilling the fermented corn to separate the fermented corn into ethanol and whole stillage; and
a dewatering system for dewatering the whole stillage, the dewatering system comprising at least one wave separator for separating the whole stillage into liquid thin stillage and wet cake, the wave separator comprising a filter screen, wave generating elements, and a driver configured to drive movement of the wave generating elements in relation to the filter screen to induce a wavelike motion in the stillage that promotes separating of the liquid thin stillage from the wet cake by breaking surface tension and encouraging the liquid thin stillage to pass through the filter screen.

2. The process of claim 1, wherein the wave separator is configured to dewater the whole stillage without shattering or emulsifying oil droplets contained in the whole stillage.

3. The process of claim 2, wherein the dewatering system is configured to dewater the whole stillage without shattering or emulsifying oil droplets contained in the whole stillage.

4. The process of claim 1, wherein the wave separator is configured to output thin stillage containing primarily settleable solids instead of floatable solids.

5. The process of claim 1, wherein the dewatering system is configured to output thin stillage containing primarily settleable solids instead of floatable solids.

6. The process of claim 1, wherein the dewatering system is configured to dewater the whole stillage without centrifuging any of the whole stillage.

7. The process of claim 1, wherein the dewatering system comprises a level sensing system configured to detect a level of stillage in the wave separator.

8. The process of claim 7, further comprising a flow control valve between the still and the wave separator, the dewatering system comprising a flow controller configured to control the flow control valve based on the level of stillage in the wave separator detected by the level sensing system.

9. The process of claim 8, wherein the level sensing system is configured to detect when the level of stillage in the wave separator reaches a low level threshold and a high level threshold.

10. The process of claim 9, wherein based on the level sensing system, the flow controller is configured to (i) close the flow control valve when the level sensing system detects the level of stillage in the wave separator reaches the high level threshold, (ii) partially open the flow control valve when the level sensing system detects the level of stillage in the wave separator has reached the low level threshold and not the high level threshold, and (iii) fully open the flow control valve when the level sensing system does not detect the level of stillage at the low level threshold.

11. The process of claim 10, wherein the dewatering system comprises a lid over the wave separator, the lid including first and second sensor fittings.

12. The process of claim 11, wherein the level sensing system comprises a low level contact sensor installed in the first sensor fitting and a high level contact sensor installed in the second sensor fitting, the low level contact sensor is configured to output a signal to the flow controller when the low level contact sensor contacts stillage at the low level threshold and the high level contact sensor is configured to output a signal to the flow controller when the high level contact sensor contacts stillage at the high level threshold.

13. The process of claim 11, wherein the lid encloses a space in the wave separator and wherein the process includes pollution control equipment in fluid communication with the space, the pollution control equipment configured to impart a vacuum on the space so that gasses in the space are evacuated to the pollution control equipment.

14. The process of claim 1, wherein the wave separator has an inlet end portion and an outlet end portion and a length extending from the inlet end portion to the outlet end portion, wherein the wave separator is configured to induce wavelike motion that drives the stillage lengthwise from the inlet end portion toward the outlet end portion.

15. The process of claim 14, wherein the wave separator has an outlet opening at the outlet end portion through which wet cake is dischargeable from the wave separator, the wave separator further comprising a press plate adjacent the outlet opening and an actuator configured to move the press plate to press liquid out of the wet cake through the filter screen before the wet cake is discharged through the outlet opening.

16. The process of claim 15, wherein the dewatering system further comprises a moisture sensor configured to detect an amount of moisture in the wet cake discharged through the outlet opening of the wave separator and a pressure regulator configured to adjust the actuator to control pressure imparted by the press plate based on the amount of moisture detected by the moisture sensor.

17. The process of claim 14, wherein the wave generating elements comprise sets of rotatable oval discs and the driver is configured drive rotation of the rotatable oval discs.

18. The process of claim 17, wherein the driver is a variable frequency drive motor configured to selectively adjust a speed at which the rotatable oval discs rotate.

19. The process of claim 14, further comprising an enclosed wet cake conveyor and wherein the dewatering system comprises a shroud between the outlet end portion of the wave separator to the enclosed wet cake conveyor, the shroud configured to direct wet cake discharged from the outlet end portion of the wave separator into the wet cake conveyor and isolate the wet cake from ambient environment, the shroud comprising a plurality of sample ports at spaced apart locations along a width of the wave separator and plurality of removable sampling tubes removably received in the sample ports.

20. The process of claim 14, wherein the dewatering system further comprises a whole stillage distributor at the inlet end portion of the wave separator, the whole stillage distributor comprising an inlet tube and an outlet tube transverse to the inlet tube and perpendicular to the length of the wave separator, the inlet tube fluidly connected to the still, the outlet tube located above the filter screen and extending generally parallel to the filter screen, the wave separator having a width, the outlet tube defining an outlet opening and comprising a weir below the outlet opening, the outlet opening and the weir extending widthwise along most of the width of the wave separator, the whole stillage distributor configured so that whole stillage flows into the whole stillage distributor in a first flow direction transverse to the length and width of the wave separator and forcefully impacts the outlet tube to create turbulent flow, after which time, the whole stillage backfills behind the weir until it overflows through the outlet opening at a substantially even flow rate across the majority of the width of the wave separator.

21. The process of claim 14, wherein the dewatering system comprises a stand supporting the wave separator, the stand comprising a gutter below the filter screen, the gutter having an upstream end portion below the inlet end portion of the wave separator and a downstream end portion below the outlet end portion of the wave separator, the upstream end portion of the gutter being elevated above the downstream end portion and the gutter sloping downward lengthwise from the upstream end portion to the downstream end portion, the gutter being V-shaped, and the process further comprising a thin stillage line configured to receive thin stillage from the downstream end portion of the gutter.

22. A method of making corn ethanol, the method comprising:
fermenting corn;
distilling the fermented corn to separate the fermented corn into ethanol and whole stillage;
dewatering the whole stillage in a wave separator to separate thin stillage from wet cake; and
extracting corn oil from the thin stillage.
